(19) United States
(12) Patent Application Publication
(10) Pub. No.: US 2015/0025645 A1
(43) Pub. Date: Jan. 22, 2015

(54) ARTIFICIAL KNEE JOINT

(71) Applicant: CORENTEC CO., LTD., Chungcheongnam-do (KR)

(72) Inventors: Woo Shin Cho, Seoul (KR); Oui Sik Yoo, Chungcheongnam-do (KR)

(73) Assignee: CORENTEC CO., LTD., Chungcheongnam-do (KR)

(21) Appl. No.: 14/378,832

(22) PCT Filed: Nov. 30, 2012
(86) PCT No.: PCT/KR12/10277

§ 371 Date: Aug. 14, 2014

(30) Foreign Application Priority Data
Feb. 17, 2012 (KR) 10 -2012-0016397

(51) Int. Cl. A61F 2/38

(52) U.S. Cl. CPC A61F 2/3886; A61F 2/389; A61F 2/3859
USPC 623/20.29; 623/20.28

(57) ABSTRACT

This invention relates to an artificial knee joint component capable of adjusting alignment or balancing (extension gap and flexion gap) between the tibia and the femur.

### TECHNICAL FIELD

This invention relates to an artificial knee joint capable of adjusting alignment or balancing.

### BACKGROUND ART

Among the many joints in the body, the knee joint is the junction between the tibia and the femur. The knee joint is the joint which is located between the lower end of the femur, the upper end of the tibia, and the back of the patella, and functions to bend the leg backwards at the knee. Since the knee joint is the most active joint including the role of supporting the body weight, it is the most vulnerable joint to degenerative arthritis due to the ageing of cartilage and bone tissue, and also it is the most susceptible joint to injury.

The back of the patella is covered by cartilage 4 mm to 6 mm thick. The patella moves up and down along the articular surface which is in front of the end portion of a thighbone (the femur) while the knee is bent or stretched (at the patellofemoral joint), thus improving the knee stretching force of the musculus quadriceps femoris. Pressure acting on the patellofemoral joint when a person walks on a flat ground is equal to half of his or her weight. When a person goes up the stairs, a force, which is 5-6 times as great as his or her weight acts on the patellofemoral joint. Further, when a person sitting in a squatting position stands up, a force 8 times as great as his or her weight acts on the patellofemoral joint. An articular capsule extends from the edge of the lower end of the femur to the edge of the upper end of the tibia. In addition to the above components, the medial and the lateral collateral ligaments, the cruciate ligaments of knee in the articular capsule, and other strong ligaments strengthen the connection of the bones and limit the moving direction and range of the bones.

By the way, when the pain occurs due to the worn cartilage of the knee joint cause by arthritis, and the functions thereof are degraded causing abasia and the like, the knee joint are being replaced by an artificial knee joint inserted therein.

Various prior art documents related to such an artificial knee joint have been known.

Patent Literature 1 relates to an artificial knee joint having a femur joint member which is attached to an end portion of the femur near the tibia, and a tibia joint member which is attached to an end portion of the tibia near the femur, wherein the artificial knee joint evenly disperses the stress of both the femur joint member and the tibia joint member regardless of the applied body weight or the excessive force applied during knee movement by enlarging the contact surface between the femur joint member and the tibia joint member.

Patent Literature 2 relates to an artificial knee joint having a femur joint member which is attached to an end portion of the femur near the tibia, and a tibia joint member which is attached to an end portion of the tibia near the femur, wherein the artificial knee joint is designed to perform movement similar to actual knee movement by having different curvature radii in the front side and the rear side of the groove in the upper surface as seen from the top of the bearing positioned between the femur joint member and the tibia joint member.

The artificial knee joint of the Patent Literature 2 includes a femur joint member (100) which is attached to the lower portion of the femur (1), a tibia joint member (300) which is attached to the upper portion of the tibia (3), and a bearing member (500) which is provided between the femur joint member and the tibia joint member and serves as cartilage. The femur joint member (100) includes a femur receiving part (110) for receiving the femur in the femur receiving part (110), a locking protrusion (150) which helps in coupling of the femur more firmly to the femur receiving part (110), and on its lower surface, curved contact portions 130 which are in contact with the bearing member (500) that will be described below.

The tibia joint member (300) is fitted into and secured to the upper portion of the tibia in the typical artificial knee joint, is made of a biocompatible material, and supports the bearing member (500) which will be described below.

The bearing member (500) includes the concave portions (510) which are provided on the upper portion of the bearing member and make contact with the contact portions (130) of the femur joint member (100) and front projecting part (530) and rear projecting part (540) formed in front and rear ends of the bearing member (500) when seen from the side.

Patent Literature 3 relates to an artificial knee joint having a femur joint member which is attached to an end portion of the femur near the tibia, a tibia joint member which is attached to an end portion of the tibia near the femur, and a bearing member which is positioned between the femur joint member and the tibia joint member, wherein the femur joint member includes a cam and the bearing member includes a post, thus resulting in a PS type artificial knee joint with removed posterior cruciate ligament, wherein the structure of the post of the bearing member and the cam of the femur joint member is improved so as to achieve natural movement when the femur joint member is slightly rotated on a plane of the upper surface of the bearing member, thereby minimizing the damage of the post, and more particularly, when the femur joint member is bent up to the inside of the knee through the front concave portion of the post, a more natural rolling movement is possible, and the collision with the patella positioned ahead is prevented when fully bent by a rolling movement, thereby enabling a natural bending movement.

Patent Literature 4 relates to an artificial knee joint having a femur joint member which is attached to an end portion of the femur near the tibia, a tibia joint member which is attached to an end portion of the tibia near the femur, and a bearing member which is positioned between the femur joint member and the tibia joint member, wherein a chamber is formed at the end of the posterior condyle of the femur joint member in order to prevent a ligament injury when there is a knee movement, thereby preventing the ligament injury caused by such as twisting, and rotation of the femur joint member.

Patent Literature 5 relates to an artificial knee joint, wherein a hole where a connection pin member can pass through is provided, and a plate having installed a slanted front surface and a rear surface therein is disclosed; said plate, which is for compensating the born loss, is fixed to the upper surface of the platform in addition to the initial platform thickness, thereby increasing the thickness of the femur component.

Patent Literature 1 to Patent Literature 4 are all for improving the detailed structures of the artificial knee joint, and Patent Literature 5 discloses a plate for compensating born condition i.e. born loss being discovered during surgery.

### LEADING TECHNICAL LITERATURE

### Patent Literature

[Patent Literature 1] Korea Patent No. 10-0901524

[Patent Literature 2] Korea Patent No. 10-0904087

[Patent Literature 3] Korea Patent No. 10-0930727

[Patent Literature 4] Korea Patent No. 10-0901528

[Patent Literature 5] U.S. Pat. No. 4,950,298

### DETAILED DESCRIPTION OF INVENTION

### Technical Problem

Good ligament balancing of the knee and precise alignment between the femur and the tibia is essential for preserving the functions of a leg that has undergone surgery and prolonged use thereof. The present invention is to provide a component of an artificial knee joint capable of adjusting the alignment between the femur and the tibia.

The present invention is to provide a component of an artificial knee joint capable of adjusting the balancing between the femur and the tibia, i.e. the unbalance between the extension gap and the flexion gap.

### Solution to Problem

An artificial knee joint of the present invention includes a thin plate-like pad, which is installed in at least any one of a femur joint member which is attached to an end portion of the femur near the tibia, and a tibia joint member which is attached to an end portion of the tibia near the femur, and adjusts alignment or balancing between the femur and the tibia.

Another artificial knee joint component of the present invention may include a pad comprising at least any one of the distal pad which is installed in a downward direction of said femur joint member; a posterior pad which is installed in a rear direction of said femur joint member; and a half pad which is installed in a left-right direction of the tibia joint member.

Yet another artificial knee joint component of the present invention is characterized in that coronal plane and axial plane alignments are corrected by using at least one type of said distal pad, said posterior pad or said half pad on one side.

Still another artificial knee joint component of the present invention is an artificial knee joint component capable of adjusting alignment or balancing characterized in that correction of the balancing is performed by using at least one type of said distal pad, said posterior pad or said half pad on both sides.

### Advantageous Effects of Invention

Alignment between the femur and the tibia, i.e. mechanical axis, can be corrected by using any one or more of the femoral distal pad and the tibial half pad of the present invention on one side of the artificial knee joint. For preserving functions and for a prolonged use, since 2-3 degrees of misalignment, or more than 2 mm dislocation in the extension and the flexion gap significantly degrades functions and reduces survival rate, the present invention has an effect on correcting such matters without additional bone resection or ligament laxity.

The balancing between the femur and the tibia, i.e. the displacement between the extension gap and the flexion gap, can be adjusted by using any one or more of the femoral distal pad and the tibial half pad on both sides of the artificial knee joint.

If a posterior pad i.e. a femoral posterior pad of the present invention is used on one side, the alignment of the rotational axis can be corrected; if they are inserted in both sides, the displacement between the extension gap and the flexion gap can be adjusted.

In this way, by reducing the bone resection volume and preventing excessive ligament laxity, surgery can be facilitated, thus a well-functioning knee joint can be made.

In addition, if a pad of the present invention is used in combination with the conventional augments, the restoration function of the bony defect can be enhanced therefore the amount of the bone resection volume occurring during surgery using only the conventional augments may be reduced

### BRIEF DESCRIPTION OF DRAWINGS

FIG. 1 is a view illustrating a conventional artificial knee joint;

FIG. 2 is a perspective view illustrating the state of attachment of a femur joint member and a pad;

FIGS. 3 to 5 corresponds to a side view, a plan view, and a front view illustrating the state of attachment of a femur joint member and a half pad;

FIG. 6 is a perspective view illustrating the state of attachment of a tibia joint member and a half pad;

FIGS. 7 to 9 corresponds to a side view, a plan view, and a front view illustrating the state of attachment of a tibia joint member and a pad;

FIG. 10 is a table and drawing summarizing the effects according to the number of pads and the locations thereof; and

FIG. 11 is a coordinate system for describing the position and effect of an artificial knee joint.

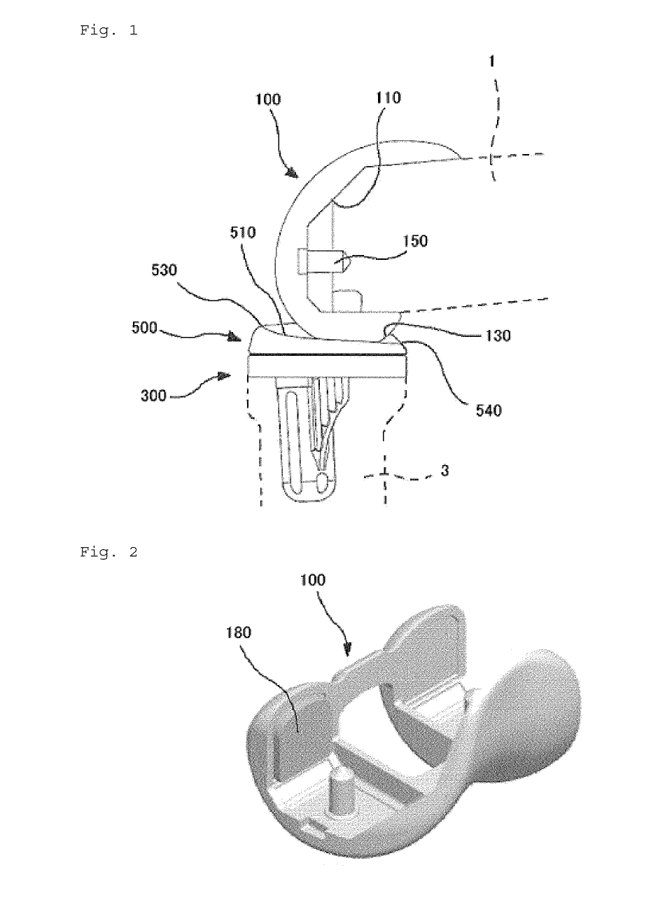

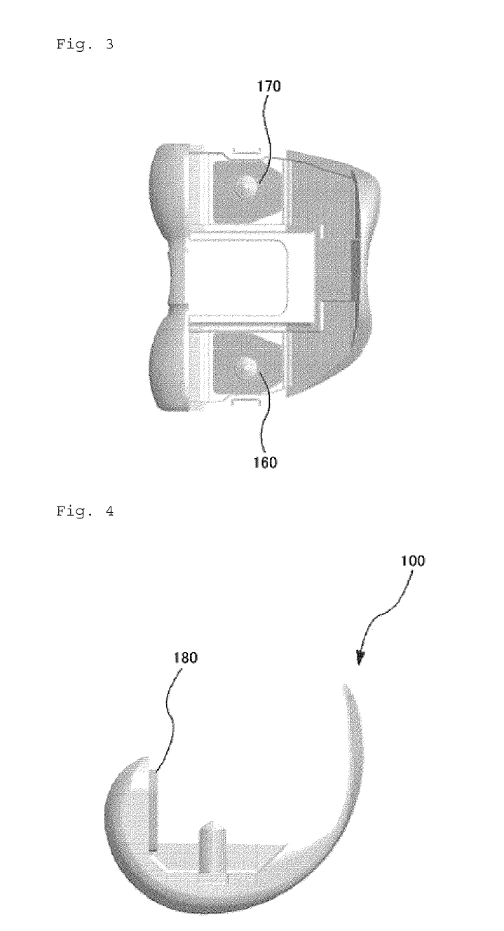

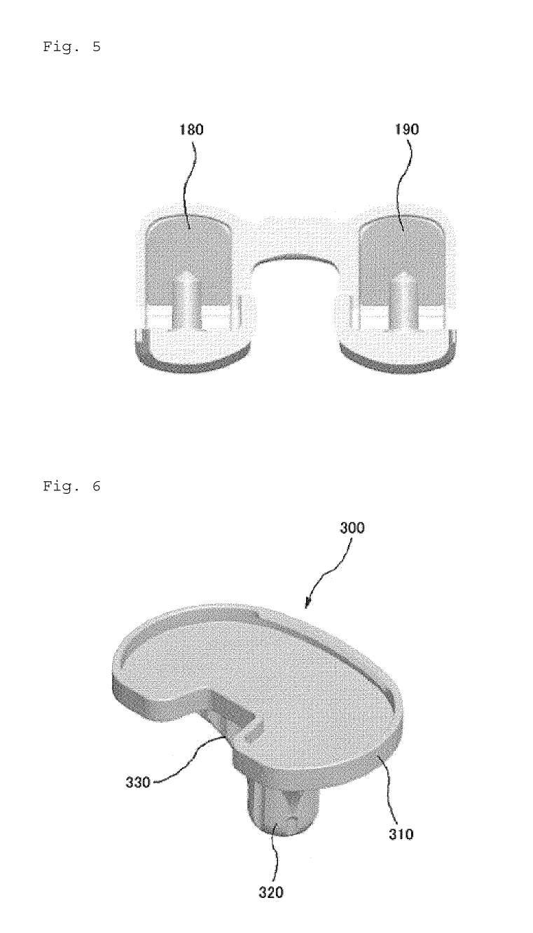

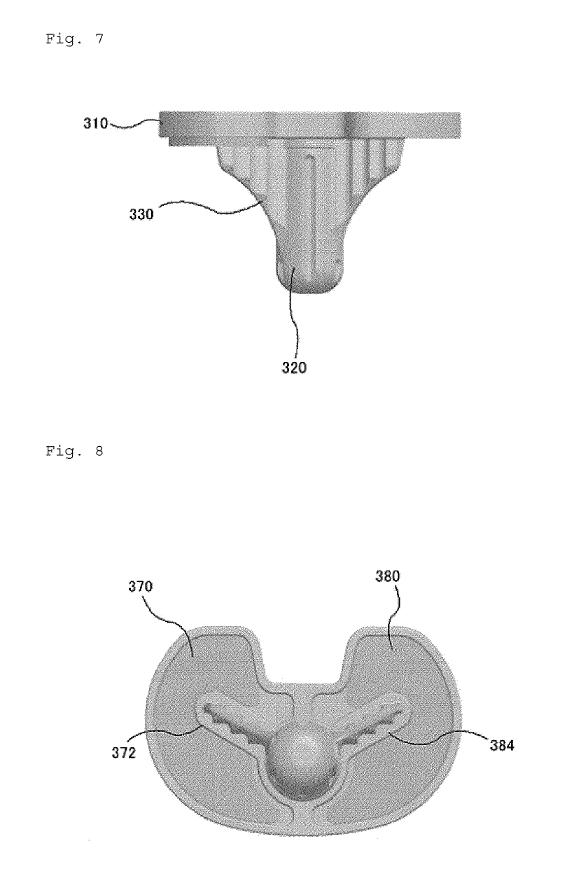

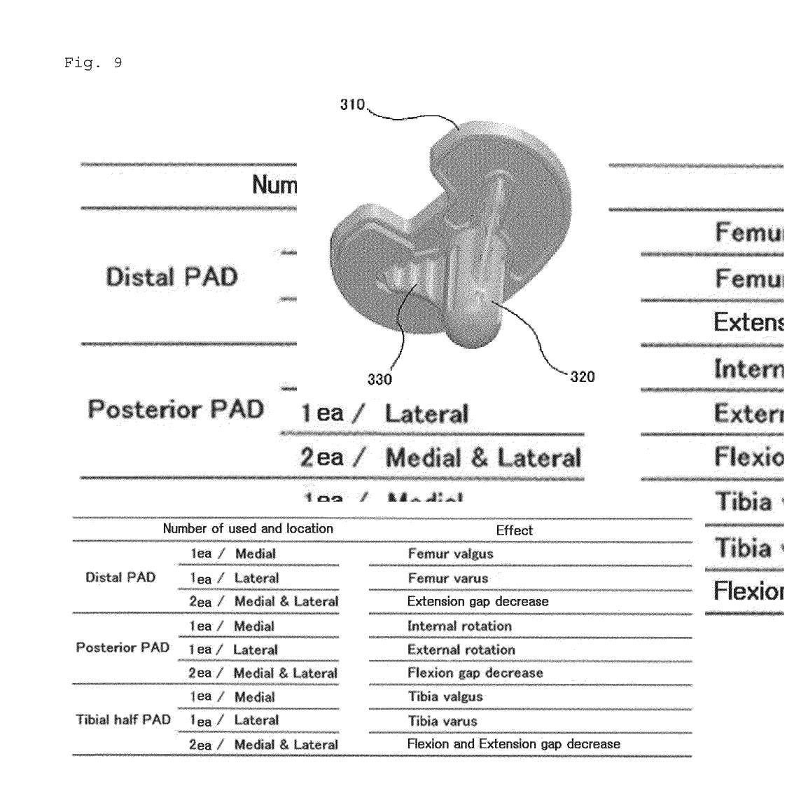

### DETAILED DESCRIPTION OF EMBODIMENT

An artificial knee joint of the present invention includes a femur joint member (100) which is attached to an end portion of the femur near the tibia; a tibia joint member (300) which is attached to an end portion of the tibia near the femur; and a thin plate-like pad (160, 170, 180, 190, 370, 380) being installed in at least any one of said femur joint member (100) and said tibia joint member (300) and adjusting alignment or balancing between the femur and the tibia.

Said pad (160, 170, 180, 190, 370, 380) comprises at least any one of: a distal pad (160, 170) whose upper surface and lower surface are installed in a downward direction of said femur joint member; a posterior pad (180, 190) whose upper surface and lower surface are installed in a rear direction of said femur joint member; and a half pad (370, 380) which is installed in a lateral direction of said tibia joint member.

[Distal Pad (160, 170)]

A hole is formed for inserting a locking protrusion (150) in each distal pad (160, 170). A distal pad (160, 170) has a rectangular shape in general. The upper surface and lower surface of the distal pad (160, 170) are installed in a downward direction of said femur joint member (100).

It is preferred that the first distal pad (160) has a trapezoidal shape and is being fixed in the groove of the femur joint member (100).

The second distal pad (170) has a rectangular shape and the front corners thereof are being cut off.

It is preferred that the shapes of the cut-off corners should be different enough in order to easily distinguish between the first distal pad (160) and the second distal pad (170)

It is also preferred that the second distal pad (170) is being fixed in the groove formed in the femur joint member (100).

In other words, the pockets, where the first distal pad (160) and the second distal pad (170) are being installed therein, have almost same widths (those of the pads are about 0.1 mm smaller) of top, bottom, left, and right sides of the above mentioned pads. In addition, the shapes of the first distal pad (160) and the second distal pad (170) are made similar to the contact portions of the pockets, thereby maximizing the lengths of the individual sides meeting the contact portions. Furthermore, since there is a risk that a pad may be slid-off from the hand of the surgeon during surgery due to the body fluid of the patient, the straight section is maximized for grabbing the pad stably. Meanwhile, self stacking is possible for certain types, and combining with the existing augments is possible for all types, thus the expandability of the usage during surgery is complemented. When only one of the first distal pad (160) and the second distal pad (170) is installed in the femur joint member (100), the alignment between the femur and the tibia can be adjusted. In other words, when only one of the first distal pad (160) and the second distal pad (170) is used, the installation width and the thickness of the pad is changed, thus the alignment is adjusted. In other words, the mechanical axis established by the femur and the tibia can be corrected.

When both of the first distal pad (160) and the second distal pad (170) are installed in the femur joint member (100), the extension gap can be corrected. In other words, when they are installed in both sides, it brings the effect of shortening the extension gap which reduces the extension gap; thus, the balancing can be achieved when there is unbalancing due to the wide extension gap during surgery.

[Posterior Pad (180, 190)]

The posterior pad (180, 190) has a shape comprising a curved portion at the top and a rectangular portion at the bottom continuously formed therein. The upper surface and lower surface of the posterior pad (180, 190) are installed along the forward-backward direction of the femur joint member (100).

It is preferred that a protrusion is formed along the outer circumference surface of the first posterior pad (180) such that the first posterior pad (180) and the second posterior pad (190) can be distinguished from each other. If the first posterior pad (180) and the second posterior pad (190) can be distinguished from each other, various shapes of surfaces or backsides maybe implemented other than the above mentioned protrusion.

The first posterior pad (180) and the second posterior pad (190) also have almost same lengths (those of the pads are about 0.1 mm smaller) of top, bottom, left, and right sides, almost same as those of the pockets, in order to insert them into the pockets and fix them therein. In addition, the lengths of the individual sides and the straight sections are maximized same as the distal pads.

When only one of the first posterior pad (180) and the second posterior pad (190) is installed in the femur joint member (100), the rotational alignment between the femur and the tibia can be adjusted, and the medial and the lateral balancing can be adjusted during bending operations.

When both of the first posterior pad (180) and the second posterior pad (190) are installed in the femur joint member (100), the flexion gap can be corrected.

[Half Pad (370, 380)]

A half pad (370, 380) is installed in the tibia joint member (300), which is attached to end portion of the tibia near said femur.

The tibia joint member (300) includes: a base (310); a column (320) which is integrally connected to the bottom of said base (310) and downwardly extended; and a connecting section (330) which integrally connects said bottom of said base (310) and the side of said column (320). On the front and the back surfaces of said connecting section (330), a multiple protrusions and grooves are successively formed in a wavy form.

A groove (372) is formed inside of the first half pad (370) wherein the connecting section (330) is to be inserted. It is preferred that the first half pad (370) is being fixed with the groove (372) being inserted into the connecting section (330).

A groove (384) is also formed inside of the second half pad (380) wherein the connecting section (330) is to be inserted. It is preferred that the second half pad (380) is being fixed with the groove (384) being inserted into the connecting section (330).

The alignment can be corrected by using any one of said first half pad (370) and the second half pad (380).

The extension gap and the flexion gap can be simultaneously corrected by using both of said first half pad (370) and the second half pad (380) simultaneously.

[Materials]

Titanium alloy or cobalt alloy is preferred as materials for a pad of the present invention, and porous pure titanium, porous titanium alloy, tantalum, and niobium could possibly be used.

[Various Combinations for Pad Usage]

The first distal pad (160), the second distal pad (170), the first posterior pad (180), the second posterior pad (190), the first half pad (370) and the second half pad (380) of the present invention, by itself or in combination with other pad, may possibly correct various types of alignment or balancing (extension gap or flexion gap).

The first distal pad (160), the second distal pad (170), the first posterior pad (180), the second posterior pad (190), the first half pad (370) and the second half pad (380) of the present invention may be used in combination with the conventional augments. In this case, since the restoration function for the bony defect works to some degree, the bone resection volume occurring during surgery can be reduced when they are used in combination with the conventional augments. In other words, since the conventional augments has only the restoration function for the bony defect and provided with thicknesses only in 5 mm, 10 mm, and 15 mm, or 4 mm, 8 mm, 12 mm, the unnecessary bone resection may occur; however, a pad of the present invention for adjusting alignments has thicknesses of 2 mm and 3 mm enabling fine adjustment, thereby preventing the unnecessary bone resection.

[Effects of the Number of the Pads Used and the Location Thereof]

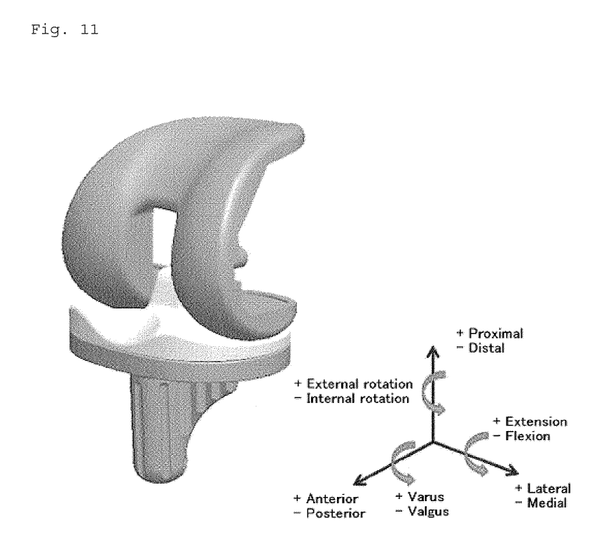

FIG. 10 summarizes the effects of the number of the pads used and the location thereof, and FIG. 11 shows a coordinate system for describing the effect of an artificial knee joint.

Using one distal pad on the medial side thereof causes effect of femur valgus; using one distal pad on the lateral side thereof causes effect of femur varus; and using two distal pads one on the medial side and the other one on the lateral side thereof causes effect of extension gap reduction.

Using one posterior pad in the medial side thereof causes effect of internal rotation; using one posterior pad in the lateral side thereof causes effect of external rotation; and using two posterior pads in both sides thereof causes effect of flexion gap reduction.

Using one half pad in the medial side thereof causes effect of tibia valgus; using one half pad in the lateral side thereof causes effect of tibia varus; and using two half pads in both sides thereof causes effect of simultaneous reduction in both the flexion gap and the extension gap.

The above mentioned effects occur in a superimposed manner according to the number of the distal pad, the posterior pad, and the half pad used therein, and the locations used therein.

### INDUSTRIAL APPLICABILITY

The present invention maybe used in all the industries wherein the artificial knee joint is needed.

### DESCRIPTION OF SYMBOLS

100: femur joint member,

300: tibia joint member,

160, 170: first distal pad, second distal pad,

180, 190: first posterior pad, second posterior pad,

370, 380: first half pad, second half pad.

What is claimed is:

1. An artificial knee joint component which includes:
a femur joint member which is attached to an end portion of the femur near the tibia;
a tibia joint member which is attached to an end portion of the tibia near the femur; and
a thin plate-like pad being installed in at least any one of said femur joint member and said tibia joint member and adjusting alignment or balancing between the femur and the tibia, wherein
said pad is characterized in that it comprises at least any one of:
a distal pad, whose upper surface and lower surface are installed in a downward direction of said femur joint member;
a posterior pad whose upper surface and lower surface are installed in a rear direction of said femur joint member; and
a half pad which are installed in a lateral direction of said tibia joint member.

2. An artificial knee joint component according to claim 1,
which is capable of adjusting the alignment and being characterized in that the extension gap is corrected by using said distal pad in both sides thereof.

3. An artificial knee joint component according to claim 1,
which is capable of adjusting the alignment and being characterized in that the flexion gap is corrected by using said posterior pad in both sides thereof.

4. An artificial knee joint component according to claim 1,
which is capable of adjusting the alignment and being characterized in that the extension gap and the flexion gap are simultaneously corrected by using said half pad in both sides thereof.
